(19) United States
(12) Patent Application Publication
(10) Pub. No.: US 2015/0182124 A1
(43) Pub. Date: Jul. 2, 2015

(54) SUBJECT INFORMATION ACQUISITION APPARATUS AND CONTROL METHOD FOR SUBJECT INFORMATION ACQUISITION APPARATUS

(71) Applicant: CANON KABUSHIKI KAISHA, Tokyo (JP)

(72) Inventor: Naoto Abe, Machida-shi (JP)

(21) Appl. No.: 14/562,887

(22) Filed: Dec. 8, 2014

(30) Foreign Application Priority Data
Dec. 27, 2013 (JP) 2013-271733

(51) Int. Cl. A61B 5/00

(52) U.S. Cl. CPC A61B 5/0095

(57) ABSTRACT

A subject information acquisition apparatus comprises a light emission unit configured to emit a pulse beam onto a subject in response to an emission trigger; an acoustic wave probe configured to receive an acoustic wave generated in an interior of the subject in response to emission of the pulse beam, and convert the received acoustic wave into an electric signal; a conversion unit configured to convert the electric signal into digital data; a clock generation unit configured to generate a sampling clock used to drive the conversion unit; an image generation unit that generates an image representing information relating to the interior of the subject on the basis of the digital data; and a synchronization unit that synchronizes the sampling clock with the emission trigger input into the light emission unit.

### BACKGROUND OF THE INVENTION

1. Field of the Invention

The present invention relates to a subject information acquisition apparatus that obtains information relating to the interior of a subject.

2. Description of the Related Art

In recent years, active research has been undertaken in the medical field into optical imaging apparatuses that turn information relating to the interior a subject into images using a photoacoustic effect.

When an organism serving as a subject is irradiated with measuring light such as a pulsed laser beam, and the measuring light is absorbed by biological tissue in the interior of the subject, an acoustic wave is generated. This phenomenon is known as the photoacoustic effect, and the acoustic wave generated by the photoacoustic effect is known as a photoacoustic wave. The tissue constituting the subject absorbs optical energy at differing absorption rates, and therefore an acoustic pressure of the generated photoacoustic wave varies. By receiving the photoacoustic wave and analyzing a reception signal, an acoustic pressure distribution of the photoacoustic wave generated in the interior of the subject can be obtained, and on the basis of this acoustic pressure distribution, an image representing information relating to the interior of the subject interior can be generated.

More specifically, the photoacoustic wave generated from the biological tissue is converted into an analog electric signal using an acoustic wave probe (a transducer), and then converted into digital data using an A/D converter. An image is then reconstructed by processing the converted digital data.

For example, Japanese Patent Application Publication No. 2012-005622 describes, as a measurement apparatus employing the photoacoustic effect, an apparatus that performs a plurality of measurements by dividing a measurement subject region into a plurality of regions, and reconstructs a single image by coupling data obtained during the respective measurements.

### SUMMARY OF THE INVENTION

When a pulse beam emission timing varies while measuring a subject using the photoacoustic effect, a data generation timing may vary such that a deviation occurs in the reconstructed image. Variation in the pulse beam emission timing is caused by jitter in a laser apparatus, for example. In the invention described in Japanese Patent Application Publication No. 2012-005622, this problem is solved by detecting the pulse beam emission timing and correcting the obtained data on the basis of the detected timing. In so doing, an image without deviations can be generated.

However, existing photoacoustic measurement apparatuses exhibit the following problem.

Since a difference between a sampling start timing of an A/D conversion circuit and the pulse beam emission timing is not fixed, a deviation occurs in the reconstructed image. This variation is smaller than the variation caused by jitter in the pulse beam, as described in Japanese Patent Application Publication No. 2012-005622. More specifically, the variation is no larger than a sampling clock period of the apparatus. Hence, the deviation in the image is likewise very small. However, an artifact caused by such a small deviation is not easily recognized as an artifact, and may lead to a misdiagnosis in the case of a medical photoacoustic imaging apparatus.

In other words, it may be said that although the deviation caused by this problem is smaller in size than a deviation that can be dealt with by the apparatus described in Japanese Patent Application Publication No. 2012-005622, an effect thereof is larger.

Further, when the difference between the sampling start timing of the A/D conversion circuit and the pulse beam emission timing varies, and the obtained data are averaged, a high frequency component decays. A method of emitting a plurality of pulse beams and averaging the data obtained as a result is often employed in a photoacoustic measurement apparatus to improve an SN ratio of the signals. However, when the difference between the sampling start timing and the pulse beam emission timing varies, or in other words when averaging processing is performed on a plurality of data having varying data heads, the high frequency component decays, leading to a reduction in measurement precision.

The present invention has been designed in consideration of this problem in the related art, and an object thereof is to provide a technique employed in a photoacoustic measurement apparatus to prevent a reduction in measurement precision caused by variation in a difference between a measuring light emission timing and a measurement data sampling start timing.

The present invention in its one aspect provides a subject information acquisition apparatus comprises a light emission unit configured to emit a pulse beam onto a subject in response to an emission trigger; an acoustic wave probe configured to receive an acoustic wave generated in an interior of the subject in response to emission of the pulse beam, and convert the received acoustic wave into an electric signal; a conversion unit configured to convert the electric signal into digital data; a clock generation unit configured to generate a sampling clock used to drive the conversion unit; an image generation unit that generates an image representing information relating to the interior of the subject on the basis of the digital data; and a synchronization unit that synchronizes the sampling clock with the emission trigger input into the light emission unit.

The present invention in its another aspect provides a control method for a subject information acquisition apparatus having a light emission unit configured to emit a pulse beam onto a subject in response to an emission trigger; and an acoustic wave probe configured to receive an acoustic wave generated in an interior of the subject in response to emission of the pulse beam, and convert the received acoustic wave into an electric signal, the control method comprises a light emission step of inputting the emission trigger into the light emission unit; a conversion step of converting the electric signal converted by the acoustic wave probe, into digital data using a sampling clock; and an image generation step of generating an image representing information relating to the interior of the subject, on the basis of the digital data, wherein the sampling clock and the emission trigger are synchronized.

According to the present invention, a reduction in the measurement precision of a photoacoustic measurement apparatus caused by variation in a difference between a measuring light emission timing and a measurement data sampling start timing can be prevented.

Further features of the present invention will become apparent from the following description of exemplary embodiments with reference to the attached drawings.

### BRIEF DESCRIPTION OF THE DRAWINGS

FIG. 1 is a view showing a configuration of a photoacoustic measurement apparatus according to the related art;

FIGS. 2A and 2B are measurement time charts according to the related art;

FIG. 3 is a second view showing the configuration of the photoacoustic measurement apparatus according to the related art;

FIGS. 4A to 4H are views illustrating decay generated in a high frequency region according to the related art;

FIG. 5 is a view showing a configuration of a photoacoustic measurement apparatus according to a first embodiment;

FIG. 6 is a measurement time chart according to the first embodiment;

FIG. 7 is a view showing a configuration of a photoacoustic measurement apparatus according to a second embodiment;

FIG. 8 is a view showing a configuration of a photoacoustic measurement apparatus according to a third embodiment;

FIG. 9 is a view showing a configuration of a photoacoustic measurement apparatus according to a fourth embodiment; and

FIG. 10 is a view showing a configuration of a photoacoustic measurement apparatus according to a modified example of the fourth embodiment.

### DESCRIPTION OF THE EMBODIMENTS

Embodiments of the present invention will be described in detail below with reference to the drawings. Note that as a rule, identical constituent elements have been allocated identical reference numerals, and description thereof has been omitted. Further, the scope of the invention is not limited to numerical values, materials, and so on used in the description of the embodiments.

### Description of Problem

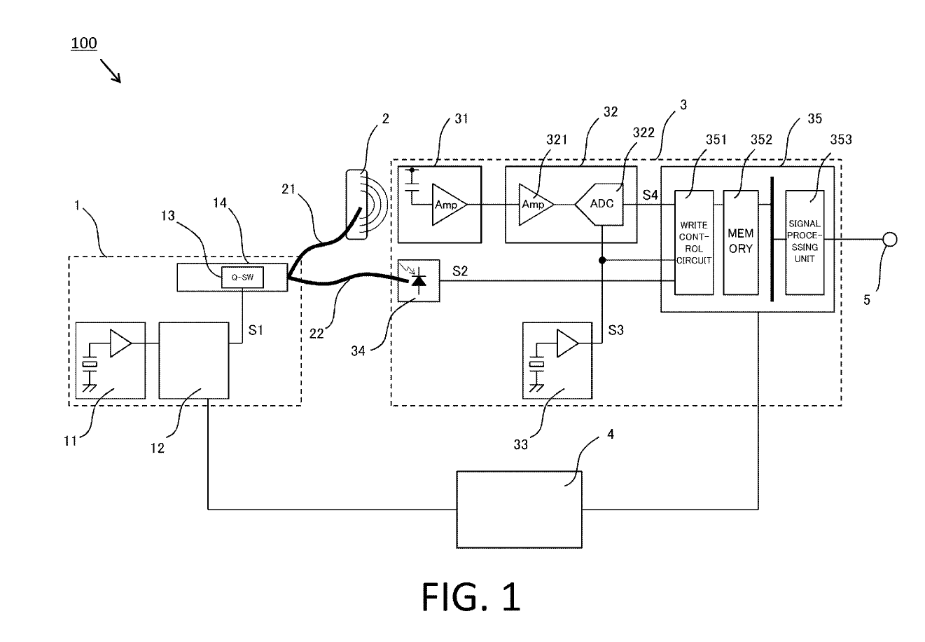

First, the problem described above will be illustrated with reference to FIG. 1, which is a system diagram showing a configuration of a photoacoustic measurement apparatus according to the related art.

A photoacoustic measurement apparatus 100 is a conventional photoacoustic measurement apparatus constituted by a laser pulse transmission unit 1, a photoacoustic reception unit 3, and a system control unit 4. Note that a reference numeral 2 denotes a subject, and a reference numeral 5 denotes an image data output terminal.

The laser pulse transmission unit 1 is a light emission unit that emits a laser pulse beam onto the subject, and is constituted by a transmission reference clock circuit 11, a laser emission control circuit 12, a Q switch 13, and a laser apparatus 14.

The transmission reference clock circuit 11 is a unit that supplies a periodic clock signal to the laser emission control circuit 12. The laser emission control circuit 12 is a circuit that transmits an emission trigger (an oscillation start signal hereafter) to the Q switch 13 to cause the Q switch 13 to generate the pulse beam.

A reference numeral 21 denotes an optical fiber that leads the laser beam to a surface of the subject, and a reference numeral 22 denotes an optical fiber used to notify the photoacoustic reception unit 3 of a laser emission timing.

The photoacoustic reception unit 3 is constituted by an ultrasonic transducer 31 that converts a photoacoustic wave into an analog electric signal (a photoacoustic signal hereafter), an AD converter 32, a reception reference clock circuit 33, a photodetector 34, and a signal processing unit 35. Further, the AD converter 32 is constituted by an amplifier 321 and an AD conversion circuit 322.

The ultrasonic transducer 31 is a unit that converts a received acoustic wave into an analog electric signal (a photoacoustic signal). The ultrasonic transducer is also known as an ultrasonic probe (an acoustic wave probe), and is constituted by a capacitance type sensor known as a CMUT or the like, for example. However, any device capable of converting an acoustic wave into an electric signal may be used. For example, a conversion element employing piezoelectric ceramics (PZT), a magnetic MUT (MMUT) employing a magnetic film, a piezoelectric MUT (PMUT) employing a piezoelectric thin film, and so on may be used.

The reception reference clock circuit 33 is a clock generation unit that generates a clock signal (a sampling clock hereafter) to drive the AD conversion circuit 322. Further, the photodetector 34 is a unit that detects a laser pulse emission timing. Furthermore, the signal processing unit 35 is an image generation unit that processes digital data (photoacoustic data hereafter) converted by the AD converter 32 so as to convert the digital data into an image. The signal processing unit 35 is constituted by a write control circuit 351, a memory 352, and a signal processing circuit 353.

When, in this apparatus, a laser emission instruction is transmitted from the system control unit 4 to the laser pulse transmission unit 1, the laser emission control circuit 12 outputs an oscillation start signal S1 to the Q switch 13 at a timing that is synchronous with the clock generated by the transmission reference clock circuit 11. In response to the signal, the laser apparatus 14 emits a laser pulse beam.

Note that in this example, the Q switch is used to perform laser oscillation, but another oscillation control unit may be used instead. For example, when a semiconductor laser is used, a sufficiently high-speed response can be obtained by direct modulation, and therefore a modulation driver may be used instead of the Q switch.

The emitted pulse beam is led to the surface of the subject by the optical fiber 21 and emitted onto the subject 2. When the subject is irradiated with the pulse beam, an acoustic wave is generated from tissue in the subject by the photoacoustic effect. This acoustic wave is received by the ultrasonic transducer 31 and converted into a photoacoustic signal. The converted photoacoustic signal is amplified to a desired amplitude by the amplifier 321, and then converted into photoacoustic data S4 by the AD conversion circuit 322.

A sampling clock S3 input into the AD conversion circuit 322 is a stable reference clock exhibiting little jitter, which is created by the reception reference clock circuit 33.

The photoacoustic data are output successively from the AD conversion circuit 322, and therefore a predetermined number of data are stored in the memory 352 at a timing set using a light reception trigger signal S2 output from the photodetector 34. In other words, the write control circuit 351 stores a predetermined number of the photoacoustic data converted by the AD conversion circuit 322 consecutively in the memory 352 in accordance with each sampling clock using a detection timing of the light reception trigger signal as a start point.

As a result, photoacoustic data corresponding to emission of the respective laser pulse beams are stored in the memory 352.

The signal processing circuit 353 then reads the photoacoustic data stored in the memory 352 and performs signal processing (image reconstruction) thereon in order to generate image data. The generated image data are output from the output terminal 5.

Note that in FIG. 1, to facilitate understanding, a configuration including the single ultrasonic transducer 31 is shown. As in Japanese Patent Application Publication No. 2012-005622, however, a plurality of photoacoustic data may be obtained simultaneously using a plurality of ultrasonic transducers. In this case, the configuration extending from the ultrasonic transducer 31 to the memory 352 is provided in a plurality, and the plurality of configurations are arranged in parallel.

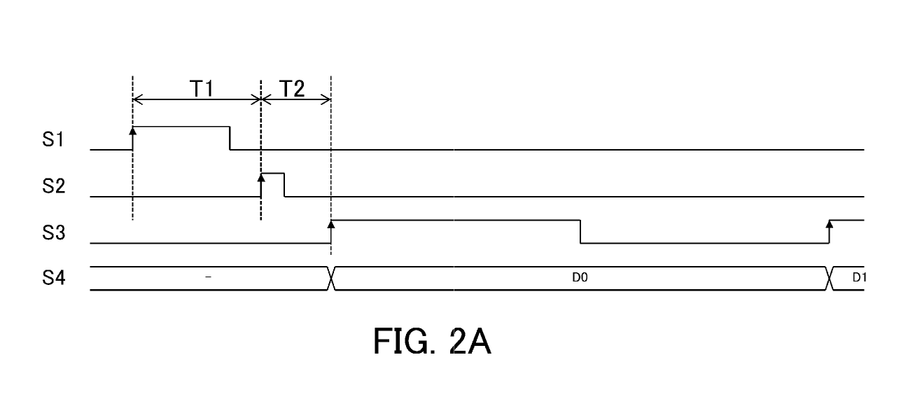

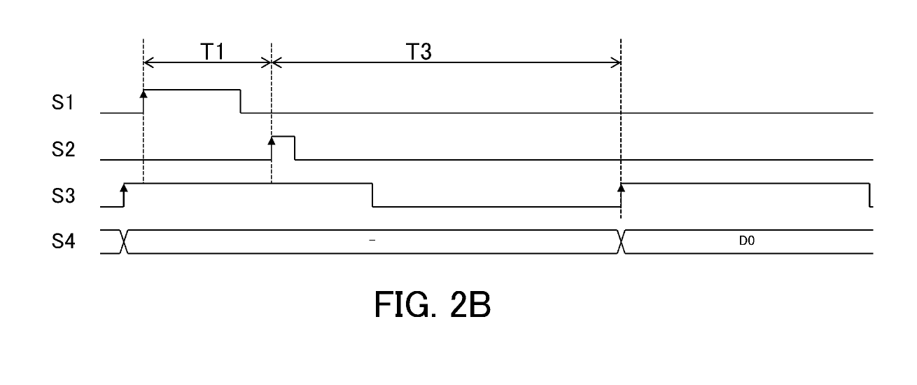

FIGS. 2A and 2B are views illustrating signal and data generation timings in the photoacoustic measurement apparatus 100 described above. In the drawings, S1, S2, S3, and S4 denote the oscillation start signal, the light reception trigger signal, the sampling clock, and the photoacoustic data, respectively.

S1 denotes the oscillation start signal input into the Q switch 13, in response to which the pulse beam is emitted. S2 denotes the light reception trigger signal output by the photodetector 34, which rises when emission of the pulse beam is detected. When the light reception trigger signal S2 is detected, the write control circuit 351 writes a predetermined number of the photoacoustic data converted by the AD conversion circuit 322 into the memory 352. In the following description, the photoacoustic data denote sampled digital data or a group (a bit string) of these data.

In the example of FIG. 2A, the light reception trigger signal S2 is generated after the elapse of a time T1 following generation of the oscillation start signal S1. In other words, the time T1 is a total time of a time extending from acquisition of the oscillation start signal S1 by the laser apparatus 14 to generation of the laser pulse, a time required for the pulse beam to propagate through the optical fiber 22, and a delay of the photodetector 34.

Further, a time T2 is a time extending from generation of the light reception trigger signal S2 to the rise of the immediately following sampling clock (in other words, a sampling start timing).

The number of data written to the memory 352 is determined from a distance specification of the measured subject in a depth direction (a Z direction). A time required for an acoustic wave generated in the interior of the subject to reach the ultrasonic transducer 31 is learned from the depth direction distance and an acoustic velocity through the subject. The number of written data can also be determined from a period of the sampling clock and this time.

A problem exhibited by a photoacoustic measurement apparatus configured as described above will now be described with reference to FIG. 2B. FIG. 2B differs from FIG. 2A in the time from generation of the light reception trigger signal S2 to the rise of the sampling clock S3.

More specifically, in FIG. 2A, photoacoustic data sampling starts after the time T2 has elapsed following the light reception trigger signal S2, whereas in FIG. 2B, sampling does not start until a time T3 has elapsed. This time difference (variation) is shorter than the period of the sampling clock S3. This variation occurs when a clock used to generate the oscillation start signal S1 and a clock used to generate the sampling clock S3 are not synchronized with each other, and the time takes a different value each time the pulse beam is emitted.

Hence, when a plurality of pulse beams are emitted, the time extending from emission of the pulse beam to the start of photoacoustic data sampling is different each time, and as a result, temporal variation occurs in the generated photoacoustic data. In other words, when the image data are generated on the basis of a plurality of photoacoustic data corresponding to the plurality of emitted pulse beams, a deviation occurs in the resulting image.

Next, decay of a high frequency component of an obtained signal will be described.

An upper limit of an amount of optical energy per unit area permitted to enter a human body is determined so that the human body is not damaged. A value of this upper limit is known as a maximum permissible exposure (MPE). In other words, when the subject is a human body, an upper level of the generated photoacoustic wave is limited.

To improve an SN ratio of a photoacoustic signal under these circumstances, a method of irradiating the subject with a plurality of pulse beams and averaging the photoacoustic data obtained as a result is conventionally employed. More specifically, a plurality of photoacoustic data are obtained by emitting a plurality of pulse beams while controlling a position of the ultrasonic transducer 31 such that the obtained photoacoustic data have identical waveforms.

Averaging may be performed by, for example, adding the obtained photoacoustic data together and dividing the result by the number of emitted pulse beams, or simply by adding the obtained photoacoustic data together. The obtained photoacoustic signals are relative, and therefore signal processing is not impaired when averaging is performed through addition alone. In this specification, the terms “average” and “averaging” are used hereafter in the description of the embodiments, but averaging may be performed by addition processing alone.

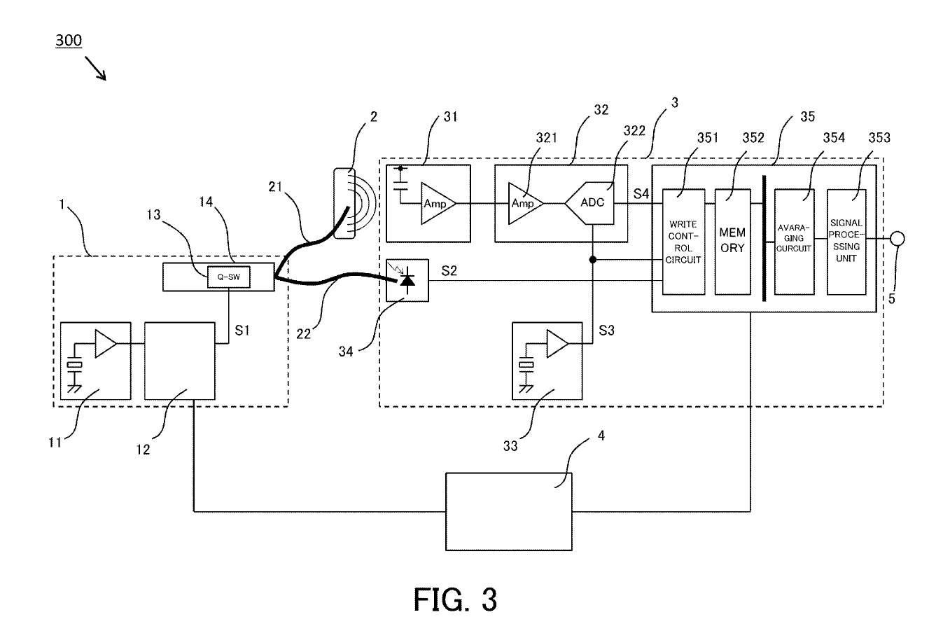

FIG. 3 is a view showing a configuration of a photoacoustic measurement apparatus 300 according to the related art, having a function for averaging obtained photoacoustic data. This apparatus is obtained by adding an averaging circuit 354 as a circuit used to average the photoacoustic data to the photoacoustic measurement apparatus 100 shown in FIG. 1.

The averaging circuit 354 is a unit that averages the photoacoustic data stored in the memory 352 (in other words, the photoacoustic data corresponding to the respective emitted pulse beam). Here, averaging is performed to average data strings of the photoacoustic data obtained during emission of the respective pulse beams. In other words, the photoacoustic data of the same order converted by the AD conversion circuit 322 each time a pulse beam is emitted are averaged by summing up the data and dividing the result using the number of emitted pulse beams.

The averaged data are then used by the signal processing circuit 353 to generate the image data.

A problem that arises during averaging of the photoacoustic data will now be described. When the photoacoustic data S4 obtained during emission of the respective laser pulses is averaged, a high frequency component of the photoacoustic data decays. The high frequency component of the photoacoustic data is a varying component that has a high frequency when the obtained photoacoustic data are arranged on a temporal axis in order of acquisition.

As described above, when a plurality of laser pulses are emitted, the position of the ultrasonic transducer 31 is controlled so that the photoacoustic data have identical waveforms. Accordingly, the photoacoustic signals corresponding to the plurality of laser pulses likewise all have identical waveforms based on the laser pulse emission timing. Naturally, when the waveforms themselves are averaged, waveform variation other than that caused by noise does not occur, and therefore signal component decay is basically absent.

However, when a plurality of pulse beams are emitted and photoacoustic data are obtained in response to each pulse beam, as described above, variation occurs between the laser pulse emission timing and the sampling start timing. In other words, the head of the obtained photoacoustic data varies such that when the photoacoustic data are averaged, a low pass filter is formed due to the variation, and as a result, the high frequency component decays.

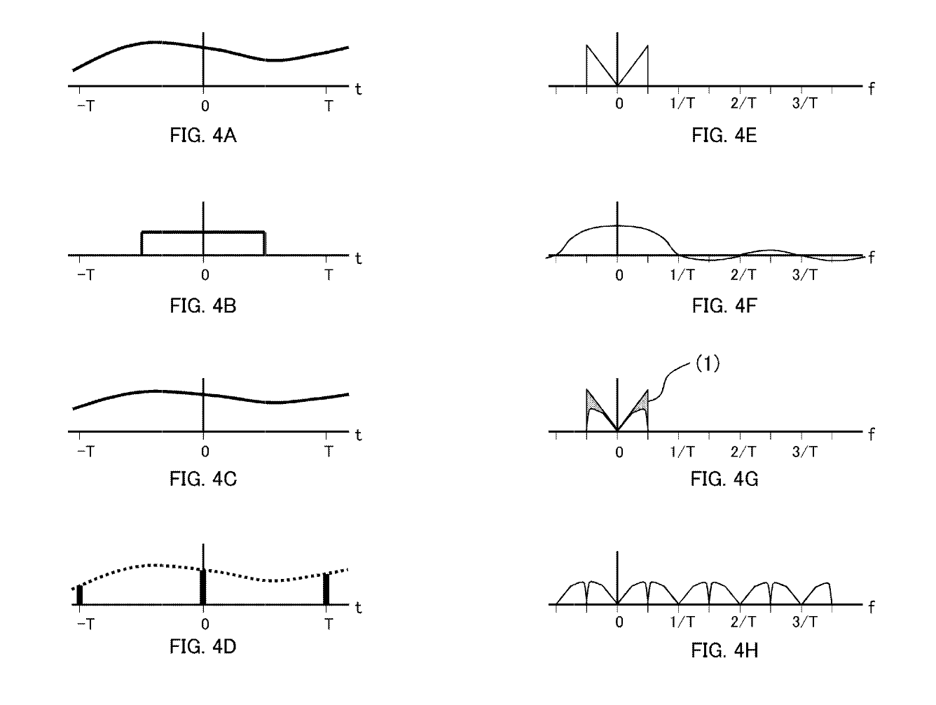

This will now be described more specifically with reference to FIGS. 4A to 4H. Here, a case in which variation between the laser pulse emission timing and the sampling start timing occurs randomly will be described as an example. FIGS. 4A to 4H are pattern diagrams showing temporal waveforms and frequency characteristics of a signal.

When a large number of laser pulses are emitted, and it is assumed that the sampling timing varies randomly, averaged photoacoustic data are obtained by adding together photoacoustic data obtained at timings varying between −T/2 and +T/2 (where T is the period of the sampling clock) each time the laser pulse is emitted, and dividing the resulting value by the number of emitted laser pulses.

In other words, the averaged photoacoustic data are equal to data (FIG. 4D) obtained by sampling a waveform shown in FIG. 4C, which is obtained by convolving an input waveform shown in FIG. 4A using a waveform shown in FIG. 4B, at intervals of the time T.

The number of emitted laser pulses is of course limited, and therefore the waveform shown in FIG. 4B is not a continuous function. When the number of emitted laser pulses is large, however, a form resembling FIG. 4B is obtained, and therefore the convolution result likewise takes a form resembling the waveform shown in FIG. 4C.

FIGS. 4E, 4F, 4G, and 4H respectively show results obtained by subjecting FIGS. 4A, 4B, 4C, and 4D to a Fourier transform. The waveform of FIG. 4C is calculated by convolving the waveforms of FIGS. 4A and 4B, and therefore, by multiplying a frequency characteristic shown in FIG. 4E by a frequency characteristic shown in FIG. 4F on a frequency axis, a frequency characteristic shown in FIG. 4G can be calculated. It can be seen that, as a result, a gray part indicated by (1) in FIG. 4G decays.

When a waveform having the frequency characteristic shown in FIG. 4G is sampled at intervals of the time T, a frequency characteristic shown in FIG. 4H is obtained. It is therefore evident that when a large number of laser pulses is emitted and the sampling timing is assumed to vary randomly, a high frequency region in the frequency characteristic of the averaged photoacoustic data decays, as shown in FIG. 4H.

In actuality, the number of emitted laser pulses may not be large and the sampling timing may not vary randomly. However, the implemented averaging processing is equivalent to a filter that adds together data obtained at varying sampling timings in each laser pulse emission, and in consideration of this fact, it is clear that although a frequency characteristic curve may take various shapes, at least the high frequency component decays. Needless to say, when data obtained at identical sampling timings are added together, a filter effect is not generated, and therefore the high frequency component does not decay.

### First Embodiment

A photoacoustic measurement apparatus according to a first embodiment visualizes, or in other words forms an image of, function information relating to an internal optical characteristic of a subject by irradiating the subject with a laser pulse beam, and then receiving and analyzing a photoacoustic wave generated in the subject in response to the pulse beam.

Furthermore, to solve the problem described above, the photoacoustic measurement apparatus according to the first embodiment has a function for synchronizing the sampling clock used during AD conversion with the clock used to emit the pulse beam.

(System Configuration)

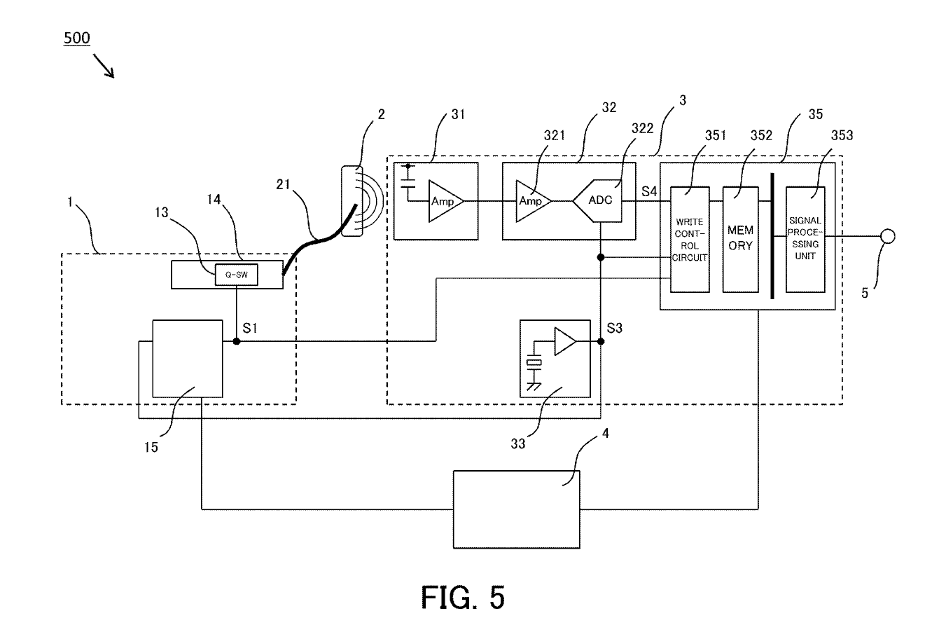

First, referring to FIG. 5, a configuration of a photoacoustic measurement apparatus 500 according to the first embodiment will be described. The photoacoustic measurement apparatus 500 according to this embodiment differs from the photoacoustic measurement apparatus 100 according to the related art in that the laser emission control circuit 12 is replaced by a laser emission synchronization control circuit 15, and the photodetector 34 is omitted.

The laser emission synchronization control circuit 15 is a unit that obtains the clock generated by the reception reference clock circuit 33, or in other words the sampling clock used during AD conversion, generates the oscillation start signal S1 at a timing that is synchronous with the sampling clock, and outputs the generated oscillation start signal S1 to the Q switch 13.

In other words, the oscillation start signal S1 is synchronous with the sampling clock S3, and therefore the sampling start timing is synchronous with the laser pulse beam emission timing. Further, the write control circuit 351 is configured to determine a timing at which to start obtaining data by obtaining the oscillation start signal S1. The write control circuit 351 obtains the photoacoustic data converted by the AD conversion circuit 322 immediately after obtaining the oscillation start signal S1, and writes a predetermined number of the obtained photoacoustic data in succession to the memory 352.

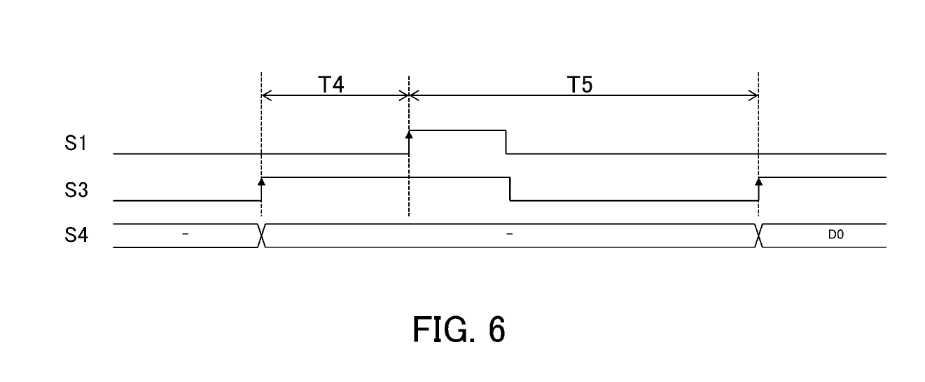

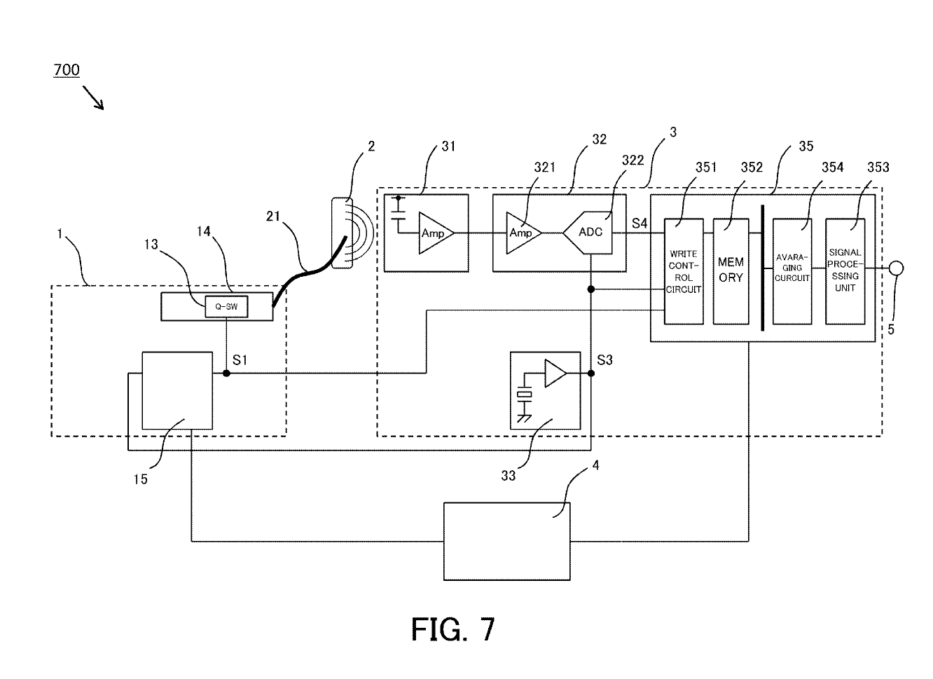

FIG. 6 is a timing chart of the first embodiment. In FIG. 6, S3 denotes the sampling clock input into the AD conversion circuit 322. In other words, conversion of the photoacoustic signal into photoacoustic data starts at the rise timing of the sampling clock.

Meanwhile, the laser emission synchronization control circuit 15 generates the oscillation start signal S1 at a timing that is synchronous with the sampling clock S3 in response to a laser emission instruction obtained from the system control unit 4, and outputs the oscillation start signal S1 to the Q switch 13. In other words, an interval between the rise timing of the sampling clock and a rise timing of the oscillation start signal S1 remains fixed (at a time T4) at all times.

The laser pulse is emitted a fixed time after the oscillation start signal S1 is input into the Q switch 13. Meanwhile, the write control circuit 351, having detected the oscillation start signal S1, stores the photoacoustic data S4 obtained on and after the detection timing in the memory 352. Storage of the photoacoustic data starts from the next rise timing of the sampling clock, and therefore a ratio between the time T4 and a time T5 in FIG. 6 remains fixed at all times.

The signal processing circuit 353 then reads the photoacoustic data stored in the memory 352, reconstructs a tomographic image, and outputs the reconstructed tomographic image from the output terminal 5.

According to the first embodiment, as described above, a deviation between the laser pulse emission timing at which each laser pulse is emitted and the sampling start timing can be kept constant.

Accordingly, a difference between the laser pulse emission timing and the photoacoustic data sampling start timing takes an identical value even when the photoacoustic data are obtained over a plurality of laser pulse emissions, and as a result, a highly precise tomographic image can be obtained. Further, in a case where measurement is performed by dividing the measurement subject region into a plurality of regions, and a plurality of images obtained as a result are synthesized, deviation among connecting parts can be prevented.

The present invention is particularly effective in a case where a plurality of measurements are performed after dividing the measurement subject region, and a plurality of image data obtained as a result are synthesized. Even when the measurement subject region is not divided, however, the Z direction of the reconstructed image data does not deviate upon each measurement, and therefore image data obtained in separate measurements can be compared easily.

Note that in the first embodiment, a circuit configuration in which the oscillation start signal S1 is input directly into the write control circuit 351 was described, but a different signal obtained in synchronization with the sampling clock S3 may be employed instead of the oscillation start signal S1. In other words, it is sufficient to ensure that the respective timings of the oscillation start signal S1 and the sampling clock S3 can be synchronized.

Further, the laser emission synchronization control circuit 15 may synchronize laser emission with a higher frequency clock generated in synchronization with the sampling clock S3. In this case, the oscillation start signal S1 may be generated at a fixed timing (phase) within the period of the sampling clock S3.

Hence, the synchronization according to the present invention may take any form as long as a temporal relationship between a rising edge (or a falling edge) of the sampling clock S3 and the oscillation start signal S1 remains constant. For example, laser emission may be synchronized with a higher frequency clock generated in synchronization with the sampling clock S3, or a fixed delay may be applied to the sampling clock S3.

### Second Embodiment

In a second embodiment, the SN ratio is improved by performing a plurality of measurements on an identical position in the interior of the subject and averaging the photoacoustic data obtained as a result.

FIG. 7 is a view showing a configuration of a photoacoustic measurement apparatus 700 according to the second embodiment. This apparatus is obtained by adding an averaging circuit 354 that averages the photoacoustic data to the photoacoustic measurement apparatus 500 according to the first embodiment. All other constituent elements are identical to the first embodiment.

In the second embodiment, similarly to the first embodiment, variation in the difference between the laser pulse emission timing and the sampling start timing can be prevented. Further, as described above, when variation occurs in the difference, the high frequency component of the photoacoustic data decays, but according to this embodiment, this decay can be prevented.

As a result, highly precise analysis can be performed on the basis of data in which the high frequency component has not decayed (in other words, non-deteriorated data). Moreover, during image reconstruction, deterioration of the image can be prevented.

Hence, the present invention can be applied favorably to a photoacoustic imaging apparatus that improves the SN ratio by emitting a plurality of laser pulses, collecting the photoacoustic data obtained during each emission, and performing averaging processing on the collected photoacoustic data.

### Third Embodiment

In the first embodiment, the oscillation start signal S1 used to emit the laser pulse beam is generated on the basis of the sampling clock generated by the reception reference clock circuit 33. In a third embodiment, on the other hand, the sampling clock S3 is generated on the basis of a transmission reference clock, which is a reference clock used to emit the laser pulse beam.

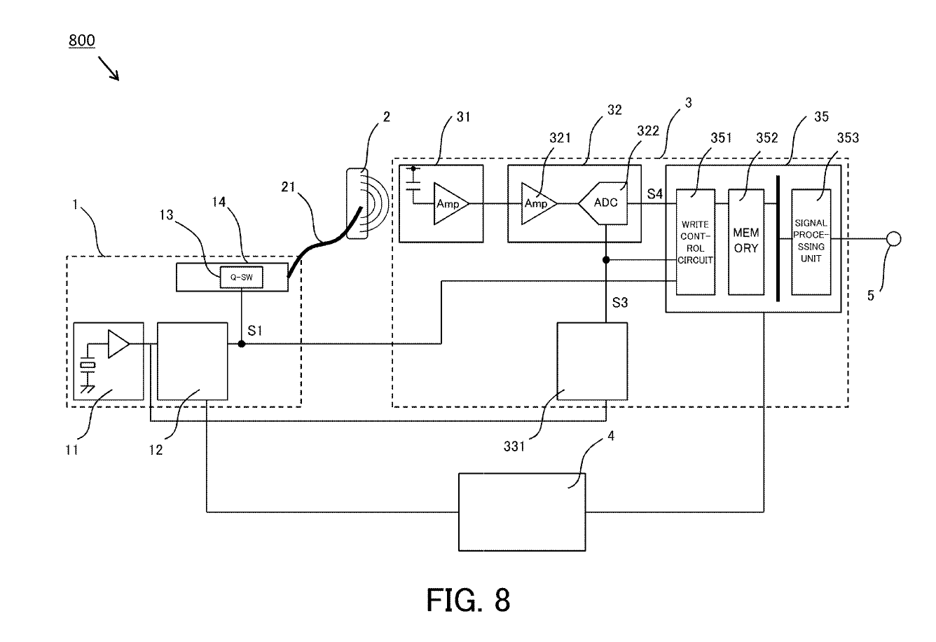

FIG. 8 is a system diagram showing a configuration of a photoacoustic measurement apparatus according to the third embodiment.

In the photoacoustic measurement apparatus according to the third embodiment, the laser emission synchronization control circuit 15 according to the first embodiment is replaced by a transmission reference clock circuit 11 and a laser emission control circuit 12, which are identical to their counterparts in the photoacoustic measurement apparatus 100. Further, the reception reference clock circuit 33 is replaced by a sampling clock generation circuit 331 that generates a sampling clock on the basis of a clock generated by the transmission reference clock circuit 11. All other units are identical to the first embodiment.

In the third embodiment, the clock (a transmission reference clock) generated by the transmission reference clock circuit 11 is used by the laser emission control circuit 12 to generate the oscillation start signal S1. Further, the transmission reference clock is input into the sampling clock generation circuit 331 and divided or multiplied in order to generate the sampling clock S3.

Likewise with this method, the oscillation start signal S1 and the sampling clock S3 can be synchronized. Note that a PLL circuit that exhibits little jitter is preferably used to divide and multiply the clock.

In the first to third embodiments, clocks are generated using the reception reference clock circuit 33 and the transmission reference clock circuit 11, but the respective clocks may be generated using another method. For example, a common clock may be generated using a common clock circuit, whereupon the oscillation start signal S1 and the sampling clock S3 may be generated respectively on the basis of the common clock.

A synchronization unit according to the present invention may be realized by a circuit having any desired configuration, as long as the sampling clock S3 and the oscillation start signal S1 can be synchronized thereby. However, a most preferable circuit configuration is that described in the first and second embodiments. In this configuration, the sampling clock S3 of the AD conversion circuit 322 is generated by the reception reference clock circuit 33 provided in the photoacoustic reception unit 3.

With this configuration, the sampling clock supplied to the AD conversion circuit 322 is generated and supplied internally. As a result, use of a buffer circuit can be reduced and clock wiring can be shortened, enabling a reduction in noise effects.

Moreover, as a result, jitter in the sampling clock S3 can be kept low. When little jitter occurs in the sampling clock S3, the SN ratio can be kept high even after increasing an AD conversion speed, and as a result, high quality photoacoustic data can be obtained.

Note that a certain amount of jitter may be added to the sampling clock S3 during transmission thereof to the laser pulse transmission unit 1. However, a certain amount of variation occurs likewise in the time extending from input of the oscillation start signal S1 into the Q switch 13 to emission of the laser pulse, and therefore the jitter occurring during transmission of the sampling clock S3 is not greatly problematic. As described above, this variation is related to the temporal deviation occurring between emission of the laser pulse and acquisition of the photoacoustic data during emission of each laser pulse, and to the reduction in the high frequency characteristic following averaging.

### Fourth Embodiment

In the first to third embodiments, a configuration in which the sampling clock S3 is synchronized with the oscillation start signal S1 was employed. In an actual photoacoustic measurement apparatus, however, the laser pulse transmission unit 1 and the photoacoustic reception unit 3 are often disposed in separate locations, and therefore instability may occur during synchronization due to the propagation time of the electric signal. In the fourth embodiment, this problem is dealt with by stabilizing synchronization using a delay circuit.

As described above with reference to FIG. 6, the oscillation start signal S1 is generated in synchronization with the sampling clock S3. When, at this time, a time between the sampling clock S3 and the oscillation start signal S1 is set as a delay time T4, T4 can be calculated as follows. The time T4 is obtained by adding together a time required for the sampling clock S3 to propagate from the photoacoustic reception unit 3 to the laser pulse transmission unit 1, a delay time of the laser emission synchronization control circuit 15, and a time required for the oscillation start signal S1 to propagate between the laser emission synchronization control circuit 15 and the write control circuit 351.

Here, no problem occurs as long as the delay time T4 takes a different value to the sampling clock period (i.e. as long as T5≅0 is not established). However, when the delay time T4 is substantially equal to the sampling clock period (i.e. when T5≅0), the write control circuit 351 may store the obtained photoacoustic data in the memory 352 at a deviation of a single sampling clock period. In this case, the photoacoustic data obtained during emission of the respective laser pulses vary.

Needless to say, even in a case where the time T4 is longer than the sampling clock period, this problem occurs likewise when the time T4 is close to an integral multiple of the sampling period. In the fourth embodiment, this problem is solved by adjusting the timings of the respective signals using a delay circuit.

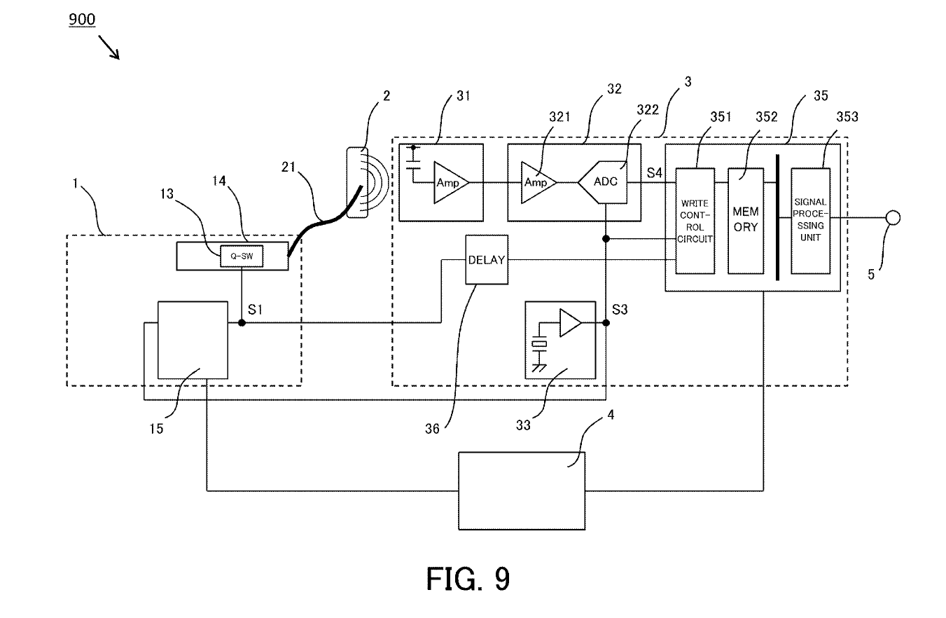

FIG. 9 is a view showing a configuration of a photoacoustic measurement apparatus 900 according to the fourth embodiment. This apparatus is obtained by adding a delay circuit 36 to the photoacoustic measurement apparatus 500 according to the first embodiment. All other constituent elements are identical to the first embodiment.

The delay circuit 36 delays an input electric signal by a predetermined time, and is capable of adjusting a delay amount. On the timing diagram shown in FIG. 6, when the delay time T4 is substantially equal to the sampling clock period (i.e. when T5≅0), the delay amount is set at half the sampling clock period, for example.

By setting the delay amount in this manner, the photoacoustic data can be obtained without causing a unit of the sampling clock period to deviate. In this case, the photoacoustic data stored in the memory 352 are data delayed by an amount corresponding to a single sampling clock period, but since this delay results only in offset of the Z direction on the image, no problems occur in the reconstructed image itself.

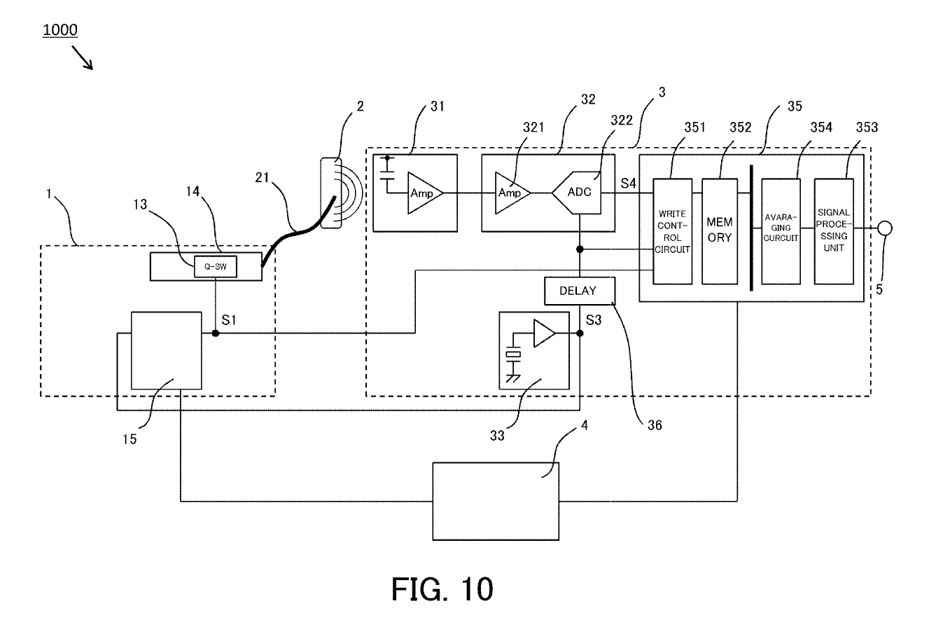

Note that in FIG. 9, the oscillation start signal S1 is delayed, but as shown in FIG. 10, the sampling clock S3 may be delayed instead.

A case in which the delay time T4 is substantially equal to the sampling clock period was described above, but likewise when the rise timing of the sampling clock S3 is identical to the timing of the oscillation start signal S1, either one of these timings may be delayed using a similar method.

FIG. 9 shows an example in which the delay circuit 36 is added to the first embodiment, while FIG. 10 shows an example in which the delay circuit 36 is added to the second embodiment. However, the delay circuit may be added to either embodiment. Moreover, a configuration in which both signals can be delayed may be provided. In other words, one or both of the sampling clock S3 and the oscillation start signal S1 may be delayed.

Furthermore, in the description of this embodiment, the signal is delayed by a time corresponding to half the sampling clock, but the delay time may be set at another value. For example, the delay time may be ⅓ or ⅔ of the sampling clock. As long as operations of the apparatus can be stabilized, the delay time may be set as desired.

According to the fourth embodiment of the present invention, as described above, by delaying at least one of the sampling clock S3 and the oscillation start signal S1, the problem whereby the photoacoustic data deviates by an amount corresponding to the unit of the sampling clock period can be solved.

Further, the delay amount can be adjusted, and therefore a modification of the distance between the laser pulse transmission unit 1 and the photoacoustic reception unit 3 can be dealt with simply by adjusting the delay amount. In other words, identical hardware can be used regardless of the location in which the apparatus is disposed.

(Modifications)

The respective embodiments are examples used for explaining the present invention, and the present invention can be implemented by appropriately changing or combining the respective embodiments without departing from the spirit of the present invention. For example, the present invention can be implemented as a method of controlling the subject information acquisition apparatus, including at least a portion of the processes. The processes and units can be freely combined with each other unless such combinations incur technical conflicts.

For example, in the description of the embodiments, the output terminal 5 that outputs the reconstructed image data is cited as an example, but a network input/output terminal, a non-volatile memory that simply stores images, or the like may be provided as an output unit. Further, in a case where a plurality of measurements are performed on divided regions and image data generated in the respective regions are coupled, a unit that couples the generated image data may be provided on the exterior of the output terminal 5.

Furthermore, in the description of the embodiments, a configuration that performs processing using hardware is cited as an example of the signal processing circuit, but the signal processing may be performed using software instead. Likewise when a unit that couples the reconstructed image data is used, processing may be performed using software. In particular, when a recent multi-core CPU is used, image reconstruction can be performed comparatively quickly.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-271733, filed on Dec. 27, 2013, which is hereby incorporated by reference herein in its entirety.

What is claimed is:

1. A subject information acquisition apparatus comprising:
a light emission unit configured to emit a pulse beam onto a subject in response to an emission trigger;
an acoustic wave probe configured to receive an acoustic wave generated in an interior of the subject in response to emission of the pulse beam, and convert the received acoustic wave into an electric signal;
a conversion unit configured to convert the electric signal into digital data;
a clock generation unit configured to generate a sampling clock used to drive the conversion unit;
an image generation unit that generates an image representing information relating to the interior of the subject on the basis of the digital data; and
a synchronization unit that synchronizes the sampling clock with the emission trigger input into the light emission unit.

2. The subject information acquisition apparatus according to claim 1, wherein the light emission unit emits a plurality of the pulse beams onto the subject,
the apparatus further comprising an averaging unit configured to collect digital data corresponding to acoustic waves generated from an identical position in the interior of the subject in accordance with emission of the plurality of pulse beams, and average the digital data.

3. The subject information acquisition apparatus according to claim 1, wherein the synchronization unit generates the emission trigger at a timing that is synchronous with the clock generated by the clock generation unit, and inputs the generated emission trigger into the light emission unit.

4. The subject information acquisition apparatus according to claim 1, further comprising a delay unit configured to delay at least one of the sampling clock used to drive the conversion unit and the emission trigger input into the light emission unit.

5. The subject information acquisition apparatus according to claim 1, wherein the light emission unit emits a plurality of the pulse beams onto the subject, and
the image generation unit generates the image by synthesizing digital data corresponding to acoustic waves generated from different positions in the interior of the subject in accordance with emission of the plurality of pulse beams.

6. A control method for a subject information acquisition apparatus having:
a light emission unit configured to emit a pulse beam onto a subject in response to an emission trigger; and
an acoustic wave probe configured to receive an acoustic wave generated in an interior of the subject in response to emission of the pulse beam, and convert the received acoustic wave into an electric signal,
the control method comprising:
a light emission step of inputting the emission trigger into the light emission unit;
a conversion step of converting the electric signal converted by the acoustic wave probe, into digital data using a sampling clock; and
an image generation step of generating an image representing information relating to the interior of the subject, on the basis of the digital data,
wherein the sampling clock and the emission trigger are synchronized.

7. The control method for a subject information acquisition apparatus according to claim 6, wherein a plurality of the pulse beams are emitted in the light emission step,
the control method further comprising an averaging step of collecting digital data corresponding to acoustic waves generated from an identical position in the interior of the subject in accordance with emission of the plurality of pulse beams, and averaging the digital data.

8. The control method for a subject information acquisition apparatus according to claim 6, wherein, in the light emission step, the emission trigger is generated at a timing that is synchronous with the sampling clock, and input into the light emission unit.

9. The control method for a subject information acquisition apparatus according to claim 6, further comprising a delaying step of delaying at least one of the sampling clock and the emission trigger input into the light emission unit.

10. The control method for a subject information acquisition apparatus according to claim 6, wherein a plurality of the pulse beams are emitted in the light emission step, and
in the image generation step, the image is generated by synthesizing digital data corresponding to acoustic waves generated from different positions in the interior of the subject in accordance with emission of the plurality of pulse beams.
